(19) United States
(12) Patent Application Publication
(10) Pub. No.: US 2008/0292699 A1
(43) Pub. Date: Nov. 27, 2008

(54) SOLID PHARMACEUTICAL DOSAGE UNIT FOR ALLEVIATING SYMPTOMS OF RHINORRHEA

(75) Inventors: Ralph BROWN, Southlake, TX (US); David BROWN, Colleyville, TX (US); Suresh DIXIT, Fort Worth, TX (US)

Correspondence Address: GREENBLUM & BERNSTEIN, P.L.C., 1950 ROLAND CLARKE PLACE, RESTON, VA, 20191, US

(73) Assignee: SOVEREIGN PHARMACEUTICALS, LTD., Fort Worth, TX (US)

(21) Appl. No.: 11/753,621

(22) Filed: May 25, 2007

(51) Int. Cl. A61K 9/24; A61K 31/439; A61K 31/137; A61K 31/435; A61P 43/00

(52) U.S. Cl. 424/468; 514/291; 514/653; 514/646; 514/357

(57) ABSTRACT

A solid pharmaceutical dosage unit for alleviating the symptoms of rhinorrhea. The dosage unit comprises an anticholinergic agent and an antihistamine and, optionally, a decongestant and, when placed in a basket in 500 ml of 0.01 N HCl of 37° C. which is stirred at 100 rpm, releases at least about 75% of the at least one anticholinergic agent within 45 minutes and releases the at least one antihistamine at a rate of from about 20% to about 60% after 2 hours, from about 45% to about 80% after 4 hours and at least about 75% after 8 hours. This Abstract is not intended to define the invention disclosed in the specification, nor intended to limit the scope of the invention in any way.

### BACKGROUND OF THE INVENTION

1. Field of the Invention

The present invention relates to a solid pharmaceutical dosage unit for alleviating symptoms of rhinorrhea. The dosage unit comprises an anticholinergic agent and an antihistamine and, optionally, a decongestant and is capable of reducing or even eliminating an over-drying of the mucous membranes caused by a combined administration of these drugs. The present invention also relates to a process for manufacturing the dosage unit and to methods for alleviating conditions which can be alleviated by a combined administration of an anticholinergic agent and an antihistamine.

2. Discussion of Background Information

Excessive thin watery mucus discharge from the nose is often the presenting symptom when patients seek medical attention for common, acute or chronic respiratory disorders. Other symptoms that usually accompany this medical disorder (rhinorrhea) are weeping eyes, sneezing, and swollen and congested mucous membranes of the nose and contiguous areas of the upper respiratory tract.

A combination of an antihistamine, a sympathomimetic decongestant, and an anticholinergic agent are the agents of choice for many physicians in controlling rhinorrhea and the underlying allergic or viral reactions that have precipitated these symptoms.

The products currently available on the market which contain a decongestant, an antihistamine and a drying (anticholinergic) agent such as methscopolamine usually cause excessive drying of the mucous membranes. More specifically, this over-drying is due primarily to the potent drying action of the anticholinergic agent combined with the weaker but still effective drying action of the antihistamine over substantially the entire dosing cycle.

It would be desirable to have available a dosage form which effectively alleviates the symptoms of rhinorrhea and at the same time is improved with respect to an over-drying of the mucous membranes that is associated with currently available dosage forms.

### SUMMARY OF THE INVENTION

The present invention provides a solid pharmaceutical dosage unit for alleviating symptoms of rhinorrhea. The dosage unit comprises at least one anticholinergic agent and at least one antihistamine and releases at least about 75% of the at least one anticholinergic agent within 45 minutes and releases the at least one antihistamine at a rate of from about 20% to about 60% after 2 hours, from about 45% to about 80% after 4 hours and at least about 75% after 8 hours when the dosage unit is placed in a basket in 500 ml of 0.01 N HCl of 37° C. which is stirred at 100 rpm.

In one aspect, the dosage unit may release at least about 80%, e.g. at least about 85%, at least about 90%, at least about 95%, or even substantially all (about 100%) of the at least one anticholinergic agent within 45 minutes.

In another aspect, the dosage unit may release the at least one antihistamine at a rate of from about 25% to about 50%, e.g., from about 30% to about 40%, or not more than about 35% after 2 hours and/or from about 50% to about 70%, e.g., from about 55% to about 60% after 4 hours and/or at least about 80%, e.g., at least about 85% after 8 hours.

In yet another aspect, the dosage unit may be a tablet, e.g., a multilayered (e.g., a bi-layered or tri-layered) tablet.

In a still further aspect, the dosage unit may provide a plasma concentration within a therapeutic range of the at least one anticholinergic agent for a first period which is not longer than about 4 hours (but preferably is at least about 2 hours), and a plasma concentration within a therapeutic range of the at least one antihistamine for a second period which is coextensive with at least about 30%, e.g., at least about 50%, at least about 70% or at least about 90% of the first period and extends at least about 2 hours, e.g., a least about 4 hours, at least about 6 hours, at least about 8 hours, or at least about 10 hours beyond the end of the first period.

In another aspect of the dosage unit, the at least one anticholinergic agent may comprise one or more of scopolamine, methscopolamine, glycopyrrolate, atropine, neostigmine and physostigmine (including pharmaceutically acceptable salts thereof). For example, the at least one anticholinergic agent may comprise methscopolamine, e.g., from about 1 mg to about 3 mg, or from about 1.5 mg to about 2.5 mg, of methscopolamine nitrate or an equivalent molar amount of another methscopolamine compound.

In another aspect of the dosage unit, the at least one antihistamine may comprise one or more of astemizole, azatadine, azelastine, bromodiphenhydramine, brompheniramine, carbinoxamine, cetirizine, chlorcyclizine, clemastine, chlorothen, chlorpheniramine, cyclizine, cyproheptadine, desloratadine, dexbrompheniramine, dimethindene, diphenhydramine, diphenylpyraline, doxylamine, fexofenadine, hydroxyzine, isothipendyl, loratadine, methapyrilene, montelukast, phenindamine, pheniramine, phenyltoloxamine, promethazine, prophenpyridamine, pyrilamine, terfenadine, thenyldiamine, thonzylamine, trimeprazine, tripelennamine and triprolidine (including pharmaceutically acceptable salts thereof). For example, the dosage unit may comprise one or more of promethazine, diphenhydramine, chlorpheniramine and carbinoxamine. By way of non-limiting example, the dosage unit may comprise chlorpheniramine, e.g., from about 9 mg to about 15 mg, for example, from about 11 mg to about 13 mg, of chlorpheniramine maleate or an equivalent molar amount of another chlorpheniramine compound.

In another aspect, the dosage unit may further comprise at least one decongestant. For example, the dosage unit, when placed in a basket in 500 ml of 0.01 N HCl of 37° C. which is stirred at 100 rpm, may release the at least one decongestant at a rate of from about 20% to about 60%, e.g., from about 25% to about 55%, from about 30% to about 50% or from about 35% to about 45% after 2 hours and/or from about 45% to about 80%, e.g. from about 55% to about 75%, or from about 65% to about 70% after 4 hours and/or at least about 75%, e.g., at least about 85% or at least about 90% after 8 hours.

In another aspect of this dosage unit, the at least one decongestant may comprise phenylephrine and/or pseudoephedrine (including pharmaceutically acceptable salts thereof), preferably at least phenylephrine. For example, the dosage unit may comprise from about 30 mg to about 50 mg, e.g., from about 35 mg to about 45 mg, of phenylephrine hydrochloride or an equivalent molar amount of another phenylephrine compound.

In another aspect, the dosage unit may provide a plasma concentration within the therapeutic range of the anticholinergic agent for a first period of from about 2 hours to about 4 hours and/or may provide a plasma concentration within the therapeutic range of the at least one antihistamine for a second period and a plasma concentration within the therapeutic range of the at least one decongestant, if present, for a third period where the second and third periods independently are coextensive with at least about 50% of the first period and extend at least about 2 hours beyond the end of the first period. By way of non-limiting example, at least one of the second and third periods may extend at least about 4 hours beyond the end of the first period and/or may be at least about 8 hours long and/or be coextensive with at least about 70% of the first period.

In another aspect of the dosage unit, the antihistamine may be present in a sustained release form and the decongestant, if present, may also be present in a sustained release form.

In another aspect of the dosage unit, the at least one anticholinergic agent may comprise methscopolamine and the dosage unit may provide a plasma concentration within the therapeutic range of the methscopolamine for a first period of from about 2 hours to about 4 hours and a plasma concentration within the therapeutic range of the at least one antihistamine for a second period and a plasma concentration within a therapeutic range of the at least one decongestant for a third period and the second and third periods may independently be coextensive with at least about 70% of the first period and extend at least about 4 hours beyond the end of the first period.

The present invention also provides a tablet for alleviating symptoms of rhinorrhea. The tablet comprises a methscopolamine compound, a chlorpheniramine compound and a phenylephrine compound. When placed in a basket in 500 ml of 0.01 N HCl of 37° C. which is stirred at 100 rpm, the tablet releases at least about 75% (e.g., at least about 80% or at least about 90%) of the methscopolamine compound within 45 minutes, releases the chlorpheniramine compound at a rate of from about 25% to about 50% after 2 hours, from about 50% to about 70% after 4 hours and at least about 80% after 8 hours, and releases the phenylephrine compound at a rate of from about 25% to about 55% after 2 hours, from about 55% to about 75% after 4 hours and at least about 80% after 8 hours.

In one aspect, the tablet may release at least about 75%, e.g., at least about 80%, at least about 90, or at least about 95% of the methscopolamine compound within 45 minutes and/or may release the chlorpheniramine compound at a rate of from about 30% to about 40% after 2 hours, and/or from about 55% to about 60% after 4 hours and/or at least about 85% after 8 hours and/or may release the phenylephrine compound at a rate of from about 30% to about 50% after 2 hours and/or from about 65% to about 70% after 4 hours and/or at least about 90% after 8 hours.

In another aspect, the tablet may comprise from about 1 mg to about 3 mg of methscopolamine nitrate or an equivalent molar amount of another methscopolamine compound, from about 9 mg to about 15 mg of chlorpheniramine maleate or an equivalent molar amount of another chlorpheniramine compound and from about 30 mg to about 50 mg of phenylephrine hydrochloride or an equivalent molar amount of another phenylephrine compound. By way of non-limiting example, the tablet may comprise from about 1.5 mg to about 2.5 mg of methscopolamine nitrate, from about 11 mg to about 13 mg of chlorpheniramine maleate and from about 35 mg to about 45 mg of phenylephrine hydrochloride.

In yet another aspect, the tablet may be a bi-layered tablet which comprises substantially the entire methscopolamine compound (e.g., at least about 95%, e.g., at least about 98%, or about 100%) in an immediate release layer and at least a part of the chlorpheniramine compound (e.g., at least about 70%, at least about 80%, or at least about 90%) and at least a part of the phenylephrine compound (e.g., at least about 70%, at least about 80%, or at least about 90%) in one or more controlled release layers (e.g., one or more sustained release layers).

The present invention also provides a bi-layered (bi-phasic) tablet for alleviating symptoms of rhinorrea which comprises a first layer and a second layer. The first layer comprises at least one anticholingeric agent and the second layer comprises at least one antihistamine. When placed in a basket in 500 ml of 0.01 N HCl of 37° C. which is stirred at 100 rpm, the tablet releases at least about 75%, e.g., at least about 80% or at least about 90% of the at least one anticholinergic agent within 45 minutes and releases the at least one antihistamine at a rate of from about 25% to about 50% after 2 hours, from about 50% to about 70% after 4 hours and at least about 80% after 8 hours.

In one aspect, the bi-layered tablet may release at least about 95% (e.g., about 100%) of the at least one anticholinergic agent within 45 minutes and/or may release the at least one antihistamine at a rate of at least about 85% after 8 hours.

In another aspect, the bi-layered tablet may further comprise at least one decongestant and may release the at least one decongestant at a rate of from about 40% to about 50% after 2 hours, from about 65% to about 75% after 4 hours and at least about 90% after 8 hours.

In yet another aspect, the bi-layered tablet may comprise an immediate release layer which comprises substantially all of the at least one anticholingeric agent and a controlled release layer which comprises at least about 80% of the at least one antihistamine and at least about 80% of the at least one decongestant.

In a still further aspect, the at least one anticholinergic agent may comprise a methscopolamine compound (i.e., methsopolamine and/or a pharmaceutically acceptable salt thereof) and/or the at least one antihistamine may comprise a chlorpheniramine compound (i.e., chlorpheniramine and/or a pharmaceutically acceptable salt thereof) and/or the at least one decongestant may comprise a phenylephrine compound (i.e., phenylephrine and/or a pharmaceutically acceptable salt thereof). By way of non-limiting example, the bi-layered tablet may comprise from about 1 mg to about 3 mg of methscopolamine nitrate or an equivalent molar amount of another methscopolamine compound, from about 9 mg to about 15 mg of chlorpheniramine maleate or an equivalent molar amount of another chlorpheniramine compound and from about 30 mg to about 50 mg of phenylephrine hydrochloride or an equivalent molar amount of another phenylephrine compound, e.g., about 2 mg of methscopolamine nitrate, about 12 mg of chlorpheniramine maleate and about 40 mg of phenylephrine hydrochloride.

In yet another aspect of the bi-layered tablet, the layers may be discrete zones which are arranged adjacent to each other, or one of the first and second layers may be partially or completely surrounded by the other one.

The present invention also provides a method of reducing or eliminating over-drying of mucous membranes in a patient which is caused by a combined administration of an anticholinergic agent and an antihistamine. The method comprises the administration to the patient of a dosage unit according to the present invention as set forth above (including the various aspects thereof).

In one aspect of the method, the at least one antihistamine may provide a therapeutic effect for at least about 8 hours and/or the at least one anticholinergic agent may provide a therapeutic effect for at least about 2 hours (but preferably not longer than about 4 hours).

The present invention also provides a method of alleviating a condition which can be alleviated by the combined administration of at least an antihistamine and an anticholinergic agent. The method comprises the administration of a dosage unit according to the present invention as set forth above (including the various aspects thereof) to a subject in need thereof.

The present invention also provides a process for making a solid pharmaceutical dosage unit of the present invention. The method comprises preparing a first composition which comprises the at least one anticholinergic agent and a second composition which comprises the at least one antihistamine, and combining the first and the second compositions. For example, the first and second compositions may be combined by using a tablet press.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those skilled in the art how the several forms of the present invention may be embodied in practice.

As set forth above, the present invention provides, inter alia, pharmaceutical dosage units for alleviating the symptoms of rhinorrhea which comprise an anticholinergic agent and an antihistamine. These dosage units may, of course, comprise more than one anticholinergic agent (e.g., two or more different anticholinergic agents and/or two or more different forms (e.g., salts) of the same anticholinergic agent) and/or more than one antihistamine (e.g., two or more different antihistamines and/or two or more different forms of the same antihistamine, for example, the free base and a pharmaceutically acceptable salt thereof, or two different pharmaceutically acceptable salts of the same parent compound). The preferred anticholinergic agent(s) for use in the present invention will have no or only an insignificant antihistaminic effect and the preferred antihistamine(s) for use in the present invention will have no or only an insignificant anticholinergic effect.

The dosage units of the present invention may also comprise one or more active ingredients which are different from anticholinergic agents and antihistamines such as, e.g., one or more active ingredients selected from decongestants, expectorants, anti-tussives, analgesics and the like. For example, the dosage forms preferably comprise at least one decongestant. Examples of preferred decongestants include phenylephrine and pseudoephedrine (including pharmaceutically acceptable salts thereof), but other decongestants may be used as well.

The active ingredients which are or may be comprised in the dosage units of the present invention, i.e., one or more anticholinergic agents, one or more antihistamines, one or more decongestants, etc., may be present as such (e.g., as free base) and/or in the form of pharmaceutically acceptable salts thereof. The term “pharmaceutically acceptable salt” as used herein refers to a salt of a particular drug that is not substantially toxic at the dosage administered to achieve the desired effect and does not independently possess significant pharmacological activity. The salts included within the scope of this term comprise, inter alia, pharmaceutically acceptable acid addition salts of suitable inorganic and organic acids. Non-limiting examples of suitable inorganic acids are, for example hydrochloric, hydrobromic, sulfuric, nitric and phosphoric acids. Non-limiting examples of suitable organic acids include carboxylic acids, such as acetic, propionic, tannic, glycolic, lactic, pyruvic, malonic, succinic, fumaric, malic, tartaric, citric, cyclamic, ascorbic, maleic, hydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acids, as well as sulfonic acids, such as methanesulfonic, ethanesulfonic and P-hydroxyethanesulfonic acids.

Non-limiting examples of drugs for use in the present invention in the form of their pharmaceutically acceptable salts include methscopolamine nitrate, methscopolamine bromide, scopolamine hydrobromide, atropine sulfate, azatadine maleate, bromodiphenhydramine HCl, brompheniramine maleate, carbinoxamine maleate, cetirizine HCl, chlorcyclizine HCl, clemastine fumarate, chlorothen citrate, chlorpheniramine maleate, dimethindene maleate, diphenhydramine HCl, fexofenadine HCl, hydroxyine HCl, isothipendyl HCl (theruhistin), methapyrilene fumarate, methapyrilene HCl, montelukast sodium, phenindamine tartrate, pheniramine maleate, phenyltoloxamine citrate, promethazine hydrochloride, prophenpyridamine maleate, pyrilamine maleate, thenyldiamine HCl, trimeprazine tartrate, tripelennamine HCl triprolidine HCl, pseudoephedrine hydrochloride and phenephrine hydrochloride, as well as codeine phosphate, codeine sulfate, hydrocodone bitartrate, dihydrocodeine bitartrate, carbetapentane citrate and guaifenesin hydrobromide.

The dosage units of the present invention may provide a plasma concentration within the therapeutic range of a particular drug (anticholinergic agent, antihistamine, decongestant, etc.) over a certain indicated period. The term “therapeutic range” as used herein and in the appended claims refers to the range of drug levels (including active metabolite levels) within which most patients will experience a significant therapeutic effect (including alleviation of symptoms) without an undesirable degree of adverse reactions when the drug is administered individually, i.e., without co-administration of other drugs.

The period over which the therapeutic range of a particular drug may be provided in a given case depends, at least in part, on the plasma half-life of the drug and/or active metabolites thereof. The plasma half-life is the time required for the plasma drug concentration to decline by 50%. The shorter the plasma half-life of a particular drug, the shorter will be the period within the therapeutic range of the drug which is provided by a single administered dose of the drug.

The dosage units of the present invention may provide a plasma concentration within the therapeutic range of an anticholinergic agent for a first period which will usually be not longer than about 4 hours (e.g., not longer than 4.5 hours), as well as a plasma concentration within the therapeutic range of an antihistamine for a second period and, optionally and preferably, a plasma concentration within the therapeutic range of a decongestant for a third period. Preferably, the second period is coextensive with at least about 30% (e.g., at least about 40%, at least about 50%, at least about 60%, at least about 70%, at least about 80%, at least about 90%, or at least about 95%) of the first period and extends at least about 2 hours (e.g., at least about 3 hours, at least about 4 hours, at least about 5 hours, at least about 6 hours, at least about 7 hours, at least about 8 hours, at least about 10 hours, or at least about 12 hours) beyond the end of the first period.

If a decongestant is present, the third period preferably is coextensive with at least about 30% (e.g., at least about 40%, at least about 50%, at least about 60%, at least about 70%, at least about 80%, at least about 90%, or at least about 95%) of the first period and extends at least about 2 hours (e.g., at least about 3 hours, at least about 4 hours, at least about 5 hours, at least about 6 hours, at least about 7 hours, at least about 8 hours, at least about 10 hours, or at least about 12 hours) beyond the end of the first period. The third period is preferably coextensive with at least about 50% of the second period, e.g., at least about 70%, at least about 80%, or at least about 90% of the second period.

In this regard, when more than one anticholinergic agent is present, the first period is calculated on the basis of the combined effect of all anticholinergic agents. By the same token, if more than one antihistamine is present, the second period is calculated on the basis of the combined effect of all antihistamines that are present. If more than one decongestant is present, the third period is calculated on the basis of the combined effect of all decongestants that are present.

It is noted that the term “coextensive with” as used herein and in the appended claims means that the second period (and the third period, where applicable) overlaps the indicated percentage of the first period. The second (third) period is longer than the indicated percentage of the first period, and at least a major part of the remainder (e.g., at least about 95% of the remainder) of the second (third) period extends beyond the end of the first period. This remainder will usually be at least about one hour, preferably at least about 2 hours. The second (third) period will usually not start before the beginning of the first period, but may start substantially at the same time as the first period (in which case the second or third period overlaps about 100% of the first period). If the second (third) period does start before the beginning of the first period, the difference in start time will usually be small, e.g., about 30 minutes or less.

The first period will preferably start within about 1 hour following the administration of the dosage unit of the present invention, for example, within about 0.7 hours, or within about 0.5 hours.

The first period will usually be not shorter than about 2 hours (e.g., not shorter than 1.5 hours). The second (third) period (including its overlap with the first period) will usually be not shorter than about 4 hours, e.g., not shorter than about 5 hours, not shorter than about 6 hours, not shorter than about 7 hours, not shorter than about 8 hours, not shorter than about 10 hours, or not shorter than about 12 hours. The second (third) period may be as long as about 24 hours, or even longer. The third period is preferably at least about as long as the second period (e.g., shorter or longer than the second period by not more than about 1 hour) and desirably substantially coextensive therewith (e.g. coextensive with at least about 90% of the second period).

In a preferred aspect of the dosage units of the present invention, the anticholinergic agent(s) is/are provided in immediate release form (e.g., in an immediate release matrix or formulation). This does not exclude, however, that a certain (usually minor) percentage of the anticholinergic agent(s) (e.g., not more than about 30%, not more than about 20%, or not more than about 10%) may be present in a controlled release form. For example, one anticholinergic agent may be present in an immediate release form and another anticholinergic agent may be present in a controlled release form, or the same anticholinergic agent may be present in both an immediate release form (major portion) and a controlled (e.g., sustained) release form.

It is usually preferred for the antihistamine(s) (and also for the decongestant(s), if used) to be present in a controlled release form (e.g., one or more controlled release matrices or formulations). This does not exclude, however, that a certain (usually minor) percentage of the antihistamine(s) (and/or of the decongestant(s)) may be present in an immediate release form (e.g., not more than about 30%, not more than about 20%, or not more than about 10%). For example, one antihistamine (decongestant) may be present in a controlled release form and another antihistamine (decongestant) may be present in an immediate release form, or the same antihistamine (decongestant) may be present in both an immediate release form (minor portion) and a controlled (e.g., sustained) release form. Also, when a decongestant is present, it may be present in the same controlled release form (e.g., the same controlled release matrix) as the antihistamine, or a different one.

If any additional active ingredients are present in the dosage units of the present invention (e.g., expectorant, anti-tussive, analgesic, etc.), these ingredients may be present in an immediate release form and/or a controlled release form and may be incorporated in the same of a different matrix than that which is used for the anticholinergic agent, antihistamine and/or decongestant.

The terms “controlled release form” and “controlled release layer” as used herein refer to any form (e.g., matrix) or layer that is not an immediate release form or layer, i.e., does not release all of the active ingredient(s) contained therein within a relatively short period (for example, within less than 1 hour, e.g., less than 0.5 hours, following administration of the dosage form). Accordingly, these terms are generic terms which encompass, e.g., sustained (extended) release forms or layers, pulsed release forms or layers, delayed release forms or layers, and the like. Preferably, the controlled release form or layer releases the one or more drugs contained therein continuously or intermittently and, preferably, in approximately equal amounts per time unit (e.g., zero order release rate), over an extended period of time such as, e.g., at least about 2 hours, at least about 3 hours, at least about 4 hours, or at least about 6 hours. The desirable length of the period of continuous or intermittent (e.g., pulsed) release depends, inter alia, on the plasma half-life of the particular drug and/or an active metabolite thereof.

A preferred, although non-limiting, embodiment of the solid dosage unit of the present invention is a tablet, in particular, a bi-layered tablet. Non-limiting examples of other embodiments of the dosage unit of the present invention are capsules, caplets, pills, and chewable tablets.

A bi-layered or bi-phasic tablet according to the present invention comprises a first layer and a second layer, with the first layer comprising an anticholingeric agent and the second layer comprising an antihistamine. This does not exclude, but rather includes, bi-layered tablets wherein a part of the anticholinergic agent(s) is present also in the second layer and/or a part of the antihistamine(s) is present also in the first layer. Preferably, the first layer comprises at least a major part, e.g., at least about 70%, at least about 80%, at least about 90%, or at least about 95% (on a molar basis) of the entire anticholinergic agent(s), and the second layer comprises at least a major part, e.g., at least about 70%, at least about 80%, at least about 90%, or at least about 95% (on a molar basis) of the entire antihistamine(s). If other active ingredients are present as well, they may be present in the first layer and/or in the second layer. In the case of the presence of one or more decongestant(s), a major part thereof, e.g., at least about 70%, at least about 80%, at least about 90%, or at least about 95% (on a molar basis), will preferably be present in the second layer.

The first layer of the bi-layered tablet will preferably be an immediate release layer and the second layer will preferably be a controlled release layer, in particular, a sustained release layer.

By way of non-limiting example and for illustrative purposes only, the bi-layered tablet of the present invention (and any other dosage unit according to the present invention) may comprise one or more of the following:

Methscopolamine: at least about 0.5 mg, e.g., at least about 0.8 mg, at least about 1 mg, at least about 1.5 mg, or at least about 1.8 mg, but not more than about 4.5 mg, e.g., not more than about 4 mg, not more than about 3.5 mg, not more than about 3 mg, or not more than about 2.5 mg, of methscopolamine nitrate, or an equivalent molar amount of any other methscopolamine compound(s) (e.g., any other pharmaceutically acceptable methscopolamine salt(s)).

Chlorpheniramine: at least about 6 mg, e.g., at least about 8 mg, at least about 10 mg, but not more than about 25 mg, e.g., not more than about 20 mg or not more than about 15 mg of chlorpheniramine maleate, or an equivalent molar amount of chlorpheniramine and/or any other pharmaceutically acceptable salt thereof.

Promethazine: at least about 1 mg, e.g., at least about 5 mg, at least about 6 mg, at least about 8 mg, at least about 12 mg, or at least about 25 mg, but not more than about 90 mg, e.g., not more than about 75 mg, not more than about 70 mg, not more than about 60 mg, or not more than about 50 mg of promethazine hydrochloride, or an equivalent molar amount of promethazine and/or any other pharmaceutically acceptable salt thereof.

Carbinoxamine: at least about 1 mg, e.g., at least about 6 mg, but not more than about 32 mg, e.g., not more than about 24 mg of carbinoxamine maleate, or an equivalent molar amount of carbinoxamine and/or any other pharmaceutically acceptable salt thereof.

Diphenhydramine: at least about 10 mg, e.g., at least about 15 mg, at least about 20 mg, at least about 40 mg, at least about 70 mg, or at least about 90 mg, but not more than about 200 mg, e.g., not more than about 150 mg, not more than about 120 mg, or not more than about 100 mg of diphenhydramine hydrochloride, or an equivalent molar amount of diphenhydramine and/or any other pharmaceutically acceptable salt thereof.

Carbetapentane: at least about 1 mg, e.g., at least about 5 mg, at least about 10 mg, at least about 25 mg, or at least about 50 mg, but not more than about 120 mg, e.g., not more than about 100 mg, not more than about 70 mg, or not more than about 60 mg, of carbetapentane citrate, or an equivalent molar amount of carbetapentane and/or any other pharmaceutically acceptable salt thereof.

Phenylephrine: at least about 10 mg, e.g., at least about 20 mg, or at least about 30 mg or at least about 40 mg, but not more than about 100 mg, not more than about 80 mg, not more than about 60 mg, or not more than about 50 mg of phenylephrine hydrochloride or an equivalent molar amount of phenylephrine and/or any other pharmaceutically acceptable salt thereof.

Pseudoephedrine: at least about 1 mg, e.g., at least about 10 mg, at least about 25 mg, or at least about 50 mg, but not more than about 240 mg, e.g., not more than about 150 mg, not more than about 100 mg, or not more than about 70 mg of pseudoephedrine hydrochloride, or an equivalent molar amount of pseudoephedrine and/or any other pharmaceutically acceptable salt thereof.

Guaifenesin: at least about 100 mg, e.g., at least about 200 mg, at least about 400 mg, at least about 600 mg, or at least about 1200 mg, but not more than about 2400 mg, e.g., not more than about 1600 mg, or not more than about 1500 mg of guaifenesin or an equivalent molar amount of a pharmaceutically acceptable salt thereof.

Codeine: at least about 1 mg, e.g., at least about 10 mg, at least about 25 mg, or at least about 30 mg, but not more than about 120 mg, e.g., not more than about 80 mg, not more than about 60 mg, or not more than about 45 mg, of codeine phosphate, or an equivalent molar amount of codeine and/or any other pharmaceutically acceptable salt thereof.

Dihydrocodeine: at least about 1 mg, e.g., at least about 5 mg, but not more than about 30 mg, e.g., not more than about 20 mg, of dihydrocodeine bitartrate, or an equivalent molar amount of dihydrocodeine and/or any other pharmaceutically acceptable salt thereof.

Hydrocodone: at least about 1 mg, e.g., at least about 5 mg, but not more than about 20 mg, e.g., not more than about 15 mg, of hydrocodone bitartrate, or an equivalent molar amount of hydrocodone and/or any other pharmaceutically acceptable salt thereof.

Acetaminophen: at least about 10 mg, e.g., at least about 50 mg, or at least about 100 mg, at least 250 mg, at least 500 mg, or at least 750 mg, but not more than about 1000 mg, e.g., not more than about 800 mg of acetaminophen.

The present invention also provides a multi-layered tablet which comprises at least a first layer and a second layer and may optionally comprise a third layer, a fourth layer, a fifth layer, etc. The first layer, which will usually be an immediate release layer, comprises one or more anticholingeric agents and the second layer, which will preferably be a controlled (e.g., sustained) release layer, comprises one or more antihistamines. As in the case of the bi-layered tablet of the present invention, this does not exclude the presence of a part of the anticholinergic agent(s) in the second layer and/or the presence of a part of the antihistamine(s) in the first layer. Alternatively or cumulatively, a part of the anticholinergic agent(s) and/or a part of the antihistamine(s) may be present in one or more additional layers (i.e., a third layer, a forth layer, etc.), e.g., in cases where two or more different different antihistamines are to be employed and it would be difficult to design a single controlled release layer which provides the desired release rates for all of these different antihistamines at the same time.

If more than two types of drugs are to be incorporated in the tablet (e.g., if one or more decongestants are to be incorporated into the tablet as well), the first and/or the second layer of the multi-layered tablet of the present invention may comprise all of the additional types of drugs. Alternatively, separate additional layers may be provided for the additional types of drugs, for example, in cases where it would be difficult to design a single controlled release layer which provides the desired release profiles for the antihistamine(s) and the additional type(s) of drug(s). Of course, a fourth, fifth, etc. layer may be provided for, e.g., a third or fourth additional type of drug, and so on. Alternatively and by way of non-limiting example, the second layer and a third layer may contain the same drug(s), but in different (relative) concentrations and/or incorporated in a different controlled release formulation.

The multi-layered tablet of the present invention will usually be made up of two or more distinct layers or discrete zones of granulation compressed together with the individual layers lying on top of one another. Layered tablets have the appearance of a sandwich because the edges of each layer or zone are exposed. Such conventional layered tablets are generally prepared by compressing a granulation onto a previously compressed granulation. The operation may be repeated to produce multi-layered tablets of more than two layers.

It is to be noted that it is not necessary for the two or more individual layers of the multi-layered tablet of the present invention to be arranged on top of one another. By way of non-limiting example, a second layer (e.g., sustained release layer) may be partially or completely surrounded by a first layer (e.g., an immediate release layer). For example, the second layer may be coated with the first layer. In the case of three layers, for example, the third layer may be partially or completely coated with the second layer, which in turn may be partially or completely coated with the first layer. Of course, these are but a few examples of the many different ways in which the various layers of the multi-layered tablet of the present invention can be arranged relative to each other. Moreover, it is to be understood that the tablets of the present invention are not limited to such multi-layered tablets. By way of non-limiting example, the tablet may comprise an immediate release matrix which comprises one or more anticholinergic agents, which matrix has dispersed therein particles of one or more sustained release formulations which have the one or more antihistamines and any of the additional desired drug(s) (e.g., one or more decongestants) incorporated therein. By way of another non-limiting example, a capsule may contain granules of the two or more different formulations.

The solid dosage units of the present invention can be manufactured by processes which are well known to those of skill in the art. For example, for the manufacture of bi-layered tablets, the active ingredients may be dispersed uniformly into a mixture of excipients, for example, by high shear granulation, low shear granulation, fluid bed granulation, or by blending for direct compression. Excipients may include diluents, binders, disintegrants, dispersants, lubricants, glidants, stabilizers, surfactants and colorants. Diluents, also termed “fillers”, are typically used to increase the bulk of a tablet so that a practical size is provided for compression. Non-limiting examples of diluents include lactose, cellulose, microcrystalline cellulose, mannitol, dry starch, hydrolyzed starches, powdered sugar, dicalcium phosphate dihydrate, calcium sulfate, calcium carbonate, alumina and kaolin. Binders impart cohesive qualities to a tablet formulation and are used to ensure that a tablet remains intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants facilitate tablet manufacture; non-limiting examples thereof include magnesium stearate, calcium stearate, stearic acid, talc, silicon dioxide glyceryl behenate, and polyethylene glycol. Disintegrants facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Non-limiting examples of suitable glidants include silicon dioxide, talc and the like. Stabilizers inhibit or retard drug decomposition reactions, including oxidative reactions. Surfactants may be anionic, cationic, amphoteric or nonionic. If desired, the tablets may also contain minor amounts of nontoxic auxiliary substances such as pH buffering agents, preservatives, e.g., antioxidants, wetting or emulsifying agents, solubilizing agents, coating agents, flavoring agents, and the like.

Extended/sustained release formulations may be made by choosing the right combination of excipients that slow the release of the active ingredients by coating or temporarily bonding or decreasing the solubility of the active ingredients. Examples of these excipients include cellulose ethers such as hydroxypropylmethylcellulose (e.g., Methocel K4M), polyvinylacetate-based excipients such as, e.g., Kollidon SR, and polymers and copolymers based on methacrylates and methacrylic acid such as, e.g., Eudragit NE 30D.

There are several commercially available tablet presses capable of making bi-layered tablets. For example, Manesty RotaPress Diamond, a 45 station D tooling press, is capable of making bi-layered tablets described in this application. Non-limiting examples of presses for the manufacture of bi-layered tablets include Fette America Model No. PT 3090; Maneklal Global Exports (Mumbai, India) Models JD and DH series; Niro Pharma Systems, Model R292F; and Korsch AG Models XL 800 and XL 400.

### EXAMPLE

A dosage form in accordance with the present invention, i.e., a bi-layered tablet, which comprises methscopolamine nitrate in an immediate release layer and chlorpheniramine maleate and phenylephrine hydrochloride in a sustained release layer is illustrated as follows:

### Sustained Release Layer:

| | | Amount per | Amount per |
|---|---|---|---|
| No. | Raw Materials | Tablet [mg] | Batch [kg] |
| | Granulating Solution I | | |
| 1 | Purified Water | 19.750 | 15.80 |
| 2 | Povidone K-30 | 6.000 | 4.800 |
| | Pre-Blend I | | |
| 3 | Prosolv SMCC 90 (Sil. Microcryst. | 30.000 | 24.000 |
| | Cellulose) | | |
| 4 | Chlorpheniramine Maleate | 12.000 | 9.600 |
| 5 | Phenylephrine HCl | 40.000 | 32.000 |
| 6 | Methocel K4M Premium | 25.000 | 20.000 |
| 7 | Povidone K-30 | 1.500 | 1.200 |
| 8 | Dibasic Calcium Phosphate Dihydrate | 10.000 | 8.000 |
| 9 | Methocel K4M | 25.000 | 20.000 |
| | Final Blend I | | |
| 10 | Microcrystalline Cellulose HD90 | 44.500 | 35.600 |
| 11 | Dye - D&C Yellow #10 Aluminum | 3.500 | 2.800 |
| | Lake (6010) | | |
| 12 | Methocel K4M Premium | 150.000 | 120.000 |
| 13 | Magnesium Stearate NF 2257 Veg | 2.500 | 2.000 |
| | Total (excluding water) | 350.000 | |

### Immediate Release Layer:

| | | Amount per | | Amount per |
|---|---|---|---|---|
| No. | Raw Materials | Tablet [mg] | | Batch [kg] |
| | Granulating Solution II | | | |
| 14 | Purified Water | 16.000 | | 12.80 |
| 15 | Methscopolamine Nitrate | 2.000 | | 1.600 |
| 16 | Povidone K-30 | 3.000 | | 2.400 |
| | Pre-Blend II | | | |
| 17 | Prosolv SMCC 90 (Sil. Microcryst. | 70.000 | | 56.000 |
| | Cellulose) | | | |
| 18 | Lactose Monohydrate NF #316 | 20.000 | | 16.000 |
| 19 | Sodium Starch Glycolate | 4.000 | | 3.200 |
| 20 | Dye - D&C Red #30 Aluminum Lake | 0.310 | | 248.3 |
| | (6030) | | | |
| 21 | Dye - D&C Blue #1 Aluminum Lake | 0.232 | | 185.6 |
| | (5516) | | | |
| | Final Blend II | | | |
| 22 | Lactose Monohydrate NF #316 | 29.884 | | 23.910 |
| 23 | Sodium Starch Glycolate | 8.000 | | 6.400 |
| 24 | Dye - D&C Red #30 Aluminum Lake | 0.328 | | 262.4 |
| | (6030) | | | |
| 25 | Dye - D&C Blue #1 Aluminum Lake | 0.246 | | 197.1 |
| | (5516) | | | |
| 26 | Prosolv SMCC 90 (Sil. Microcryst. | 60.000 | | 48.000 |
| | Cellulose) | | | |
| 27 | Magnesium Stearate NF 2257 Veg | 2.000 | | 1.600 |
| | Total (excluding water) | 200.000 | | |
| | Total Tablet Weight | 550.00 | mg | |

Procedure

Granulating Solution I:

1. Add No. 1 to a stainless steel container and slowly add No. 2 while stirring at 1000 rpm. while stirring at 1000 rpm. After all of No. 2 has been added, continue mixing for 30 minutes.

Pre-Blend I:

2. Charge Nos. 3-8 one after the other into a Lodige Mixer Model FKM300-D
3. Blend for 10 minutes with settings MIXERS ON and CHOPPERS OFF
4. With settings MIXERS ON and CHOPPERS OFF and AIR SUPPLY ON, pump Granulating Solution I to the Lodige Mixer using a Masterflex persistaltic pump model 7585-30 fitted ¾ inch tubing. Once all of the Granulating Solution I has been added, change settings to MIXERS OFF and AIR SUPPLY OFF.
5. Charge No. 9 to the Lodige Mixer, turn air supply on and blend for 2 minutes with settings MIXERS ON and CHOPPERS ON.
6. Transfer the granulation onto paper lined drying trays and dry granulation in oven for 24 hours at a temperature of from 37° C. to 53° C.
7. Pass dried granulation through a Fitzpatrick Mill (14 mesh screen) with settings HIGH SPEED and KNIVES FORWARD.

Granulation Solution II:

8. Add No. 14 (minus 0.5 kg) to a stainless steel container and slowly add No. 15 while stirring at 1000 rpm. After all of No. 15 has been added, continue mixing for 10 minutes.
9. Slowly add No. 16 while stirring at 1000 rpm. After all of No. 16 has been added, continue mixing for 30 minutes.

Pre-Blend II:

10. Charge Nos. 17-21 one after another into a Lodige Mixer Model FKM300-D
11. With setting AIR SUPPLY ON blend for 10 minutes with settings MIXERS ON and CHOPPERS OFF.
12. With settings MIXERS ON and CHOPPERS OFF and AIR SUPPLY ON, pump Granulating Solution II to the Lodige Mixer using a Masterflex persistaltic pump model 7585-30 fitted ¾ inch tubing. Once all of the Granulating Solution II has been added, change settings to MIXERS OFF and AIR SUPPLY OFF.
13. Rinse the Granulation Solution II container with remainder of No. 14 from step 8 and pump the rinse into the Lodige Mixer (settings MIXERS ON, CHOPPERS OFF, AIR SUPPLY ON).
14. With AIR SUPPLY ON blend for 2 minutes with settings MIXERS ON, CHOPPERS ON.
15. Transfer the granulation onto paper lined drying trays and dry granulation in oven for 24 hours at a temperature of from 37° C. to 53° C.
16. Pass dried granulation through a Fitzpatrick Mill (14 mesh screen) with settings HIGH SPEED and KNIVES FORWARD.

Final Blend I:

17. Pass Nos. 10-12 one after the other through an oscillating granulator (14 mesh screen) and discharge into a drum.
18. Charge milled Pre-Blend I from step 7 and the blend from step 17 into a V Blender and blend for 20 minutes.
19. Screen No. 13 through a 30 mesh hand screen, charge the screened material into V Blender, blend for 3 minutes and discharge blend into drum.

Final Blend II:

20. Pass Nos. 22-26 one after the other through an oscillating granulator (14 mesh screen) and discharge into a drum.
21. Charge milled Pre-Blend II from step 16 and the blend from step 20 into a V Blender and blend for 20 minutes.
22. Screen No. 27 through a 30 mesh hand screen, charge the screened material into V Blender, blend for 3 minutes and discharge blend into drum.

Compress Final Blend I from step 19 and Final Blend II from step 22 in a tablet press (0.2300″×0.5800 Capsule shaped) to produce capsules having a weight of about 0.550 g.

Dissolution Testing

1. Place 500 mL (+/−5 mL) of 0.01 N HCl in each vessel of the dissolution apparatus (USP I, baskets). Allow media to equilibrate to 37° C.+/−0.5° C.
2. Select 6 tablets at random and record weight of each tablet.
3. Put one tablet into each basket. Turn dissolution apparatus on to a rate of 100 rpm.
4. Withdraw samples through a 45 μm porous polyethylene filter disc (Whatman GE/X or Millipore Millex-GN) at 45 minutes, 2, 4 and 8 hours.
5. Inject filtered samples into a gas chromatograph, injecting one standard (as used for calibration) after every six samples.

Gas Chromatography:

### Instrument: Waters Alliance 2695 Separation Module

### Column: Waters μBondapak C18 Column, 10 μm, 3.9×300 mm; PN WAT027324

### Column Temperature: 52° C.

Flow Rate: 1.8 mL per minute

### Injection Volume: 200 μL

### Detector: UV at 257 nm

### Mobile Phase: A: Methanol

B: Pic-B8 (for every liter of Pic-B8 aqueous solution, dissolve 2.164 g of 1-octanesulfonic acid sodium salt in water and add 20 mL of glacial acetic acid)

Profile

| | Time (min) | A % | B % | Curve |
|---|---|---|---|---|
| Gradient: | 0.00 | 40 | 60 | N/A |
| | 10.00 | 70 | 30 | 6 |
| | 11.00 | 40 | 60 | 11 |

Run Time: 14 minutes

### Typical Retention Times:

| | |
|---|---|
| Phenylephrine HCl (PEH) | about 3.2 min. |
| Methscopolamine Nitrate (MSPN) | about 4.5 min. |
| Chlorpheniramine Maleate (CPM) | about 7.9 min. |

Stock Standard Solutions in deionized water are prepared with the following concentrations and used to calibrate the chromatograph and to make corrections for filter binding:

| | |
|---|---|
| PEH | 0.080 mg/mL |
| MSPN | 0.004 mg/mL |
| CPM | 0.024 mg/mL |

Results of Dissolution Testing (average of six tablets):

| | | |
|---|---|---|
| Average Amount Released [%] | SD | % RSD |

PEH

| | | | |
|---|---|---|---|
| Time [hr] | | | |
| 2 | 43.6 | 2.8 | 6.4 |
| 4 | 65.8 | 2.8 | 4.3 |
| 8 | 89.0 | 3.1 | 3.5 |

CPM

| | | | |
|---|---|---|---|
| 2 | 33.3 | 2.9 | 8.7 |
| 4 | 57.1 | 4.2 | 7.3 |
| 8 | 87.0 | 4.7 | 5.4 |

MSPN

| | | | |
|---|---|---|---|
| Time [min] | | | |
| 45 | 100.2 | 4.4 | 4.4 |

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A solid pharmaceutical dosage unit for alleviating symptoms of rhinorrhea, wherein the dosage unit comprises at least one anticholinergic agent and at least one antihistamine and wherein the dosage unit, when placed in a basket in 500 ml of 0.01 N HCl of 37° C. which is stirred at 100 rpm, releases at least about 75% of the at least one anticholinergic agent within 45 minutes and releases the at least one antihistamine at a rate of from about 20% to about 60% after 2 hours, from about 45% to about 80% after 4 hours and at least about 75% after 8 hours.

2. The dosage unit of claim 1, wherein the dosage unit releases at least about 85% of the at least one anticholinergic agent within 45 minutes.

3. The dosage unit of claim 1, wherein the dosage unit releases at least about 90% of the at least one anticholinergic agent within 45 minutes.

4. The dosage unit of claim 1, wherein the dosage unit releases at least about 95% of the at least one anticholinergic agent within 45 minutes.

5. The dosage unit of claim 2, wherein the dosage unit releases the at least one antihistamine at a rate of from about 25% to about 50% after 2 hours, from about 50% to about 70% after 4 hours and at least about 80% after 8 hours.

6. The dosage unit of claim 3, wherein the dosage unit releases the at least one antihistamine at a rate of from about 30% to about 40% after 2 hours, from about 55% to about 60% after 4 hours and at least about 85% after 8 hours.

7. The dosage unit of claim 6, wherein the dosage unit releases the at least one antihistamine at a rate of not more than about 35% after 2 hours.

8. The dosage unit of claim 1, wherein the dosage unit is a tablet.

9. The dosage unit of claim 8, wherein the tablet is a multilayered tablet.

10. The dosage unit of claim 1, wherein the dosage unit provides a plasma concentration within a therapeutic range of the at least one anticholinergic agent for a first period which is not longer than about 4 hours and a plasma concentration within a therapeutic range of the at least one antihistamine for a second period which is coextensive with at least about 30% of the first period and extends at least about 2 hours beyond an end of the first period.

11. The dosage unit of claim 10, wherein the second period extends at least about 4 hours beyond the end of the first period.

12. The dosage unit of claim 10, wherein the second period extends at least about 6 hours beyond the end of the first period.

13. The dosage form of claim 10, wherein the first period is at least about 2 hours long.

14. The dosage unit of claim 13, wherein the second period is at least about 6 hours long.

15. The dosage unit of claim 10, wherein the second period is at least about 10 hours long.

16. The dosage unit of claim 14, wherein the second period is coextensive with at least about 50% of the first period.

17. The dosage unit of claim 15, wherein the second period is coextensive with at least about 90% of the first period.

18. The dosage unit of claim 1, wherein the at least one anticholinergic agent comprises one or more of scopolamine, methscopolamine, glycopyrrolate, atropine, neostigmine and physostigmine.

19. The dosage unit of claim 18, wherein the at least one anticholinergic agent comprises methscopolamine.

20. The dosage unit of claim 19, wherein the dosage unit comprises from about 1 mg to about 3 mg of methscopolamine nitrate or an equivalent molar amount of another methscopolamine compound.

21. The dosage unit of claim 1, wherein the at least one antihistamine comprises one or more of astemizole, azatadine, azelastine, bromodiphenhydramine, brompheniramine, carbinoxamine, cetirizine, chlorcyclizine, clemastine, chlorothen, chlorpheniramine, cyclizine, cyproheptadine, desloratadine, dexbrompheniramine, dimethindene, diphenhydramine, diphenylpyraline, doxylamine, fexofenadine, hydroxyzine, isothipendyl, loratadine, methapyrilene, montelukast, phenindamine, pheniramine, phenyltoloxamine, promethazine, prophenpyridamine, pyrilamine, terfenadine, thenyldiamine, thonzylamine, trimeprazine, tripelennamine and triprolidine.

22. The dosage unit of claim 21, wherein the at least one antihistamine comprises one or more of promethazine, diphenhydramine, chlorpheniramine and carbinoxamine.

23. The dosage unit of claim 22, wherein the at least one antihistamine comprises chlorpheniramine.

24. The dosage unit of claim 23, wherein the dosage unit comprises from about 9 mg to about 15 mg of chlorpheniramine maleate or an equivalent molar amount of another chlorpheniramine compound.

25. The dosage unit of claim 1, wherein the dosage unit further comprises at least one decongestant.

26. The dosage unit of claim 25, wherein the dosage unit, when placed in a basket in 500 ml of 0.01 N HCl of 37° C. which is stirred at 100 rpm, releases the at least one decongestant at a rate of from about 20% to about 60% after 2 hours, from about 45% to about 80% after 4 hours and at least about 75% after 8 hours.

27. The dosage unit of claim 2, wherein the dosage unit further comprises at least one decongestant and, when placed in a basket in 500 ml of 0.01 N HCl of 37° C. which is stirred at 100 rpm, releases the at least one decongestant at a rate of from about 25% to about 55% after 2 hours, from about 55% to about 75% after 4 hours and at least about 85% after 8 hours.

28. The dosage unit of claim 6, wherein the dosage unit further comprises at least one decongestant and, when placed in a basket in 500 ml of 0.01 N HCl of 37° C. which is stirred at 100 rpm, releases the at least one decongestant at a rate of from about 30% to about 50% after 2 hours, from about 65% to about 70% after 4 hours and at least about 90% after 8 hours.

29. The dosage unit of claim 28, wherein the dosage unit releases the at least one decongestant at a rate of from about 35% to about 45% after 2 hours.

30. The dosage unit of claim 26, wherein the at least one decongestant comprises one or both of phenylephrine and pseudoephedrine.

31. The dosage unit of claim 30, wherein the at least decongestant comprises phenylephrine.

32. The dosage unit of claim 31, wherein the dosage unit comprises from about 30 mg to about 50 mg of phenylephrine hydrochloride or an equivalent molar amount of another phenylephrine compound.

33. A tablet for alleviating symptoms of rhinorrhea, wherein the tablet comprises a methscopolamine compound, a chlorpheniramine compound and a phenylephrine compound and wherein the tablet, when placed in a basket in 500 ml of 0.01 N HCl of 37° C. which is stirred at 100 rpm, releases at least about 75% of the methscopolamine compound within 45 minutes, releases the chlorpheniramine compound at a rate of from about 25% to about 50% after 2 hours, from about 50% to about 70% after 4 hours and at least about 80% after 8 hours, and releases the phenylephrine compound at a rate of from about 25% to about 55% after 2 hours, from about 55% to about 75% after 4 hours and at least about 85% after 8 hours.

34. The tablet of claim 33, wherein the tablet releases at least about 90% of the methscopolamine compound within 45 minutes, releases the chlorpheniramine compound at a rate of from about 30% to about 40% after 2 hours, from about 55% to about 60% after 4 hours and at least about 85% after 8 hours and releases the phenylephrine compound at a rate of from about 30% to about 50% after 2 hours, from about 65% to about 70% after 4 hours and at least about 90% after 8 hours.

35. The tablet of claim 34, wherein the tablet comprises from about 1 mg to about 3 mg of methscopolamine nitrate or an equivalent molar amount of another methscopolamine compound, from about 9 mg to about 15 mg of chlorpheniramine maleate or an equivalent molar amount of another chlorpheniramine compound and from about 30 mg to about 50 mg of phenylephrine hydrochloride or an equivalent molar amount of another phenylephrine compound.

36. The tablet of claim 35, wherein the tablet comprises from about 1.5 mg to about 2.5 mg of methscopolamine nitrate, from about 11 mg to about 13 mg of chlorpheniramine maleate and from about 35 mg to about 45 mg of phenylephrine hydrochloride.

37. The tablet of claim 34, wherein the tablet is a bi-layered tablet which comprises substantially the entire methscopolamine compound in an immediate release layer and at least a part of the chlorpheniramine compound and at least a part of the phenylephrine compound in a controlled release layer.

38. A bi-layered tablet for alleviating symptoms of rhinorrhea, wherein the tablet comprises a first layer and a second layer, the first layer comprises at least one anticholingeric agent and the second layer comprises at least one antihistamine and wherein the tablet, when placed in a basket in 500 ml of 0.01 N HCl of 37° C. which is stirred at 100 rpm, releases at least about 75% of the at least one anticholinergic agent within 45 minutes and releases the at least one antihistamine at a rate of from about 25% to about 50% after 2 hours, from about 50% to about 70% after 4 hours and at least about 80% after 8 hours.

39. The bi-layered tablet of claim 38, wherein the tablet releases at least about 80% of the at least one anticholinergic agent within 45 minutes and releases the at least one antihistamine at a rate of at least about 85% after 8 hours.

40. The bi-layered tablet of claim 38, wherein the tablet further comprises at least one decongestant and releases the at least one decongestant at a rate of from about 40% to about 50% after 2 hours, from about 65% to about 75% after 4 hours and at least about 90% after 8 hours.

41. The bi-layered tablet of claim 40, wherein the tablet comprises an immediate release layer which comprises substantially all of the at least one anticholingeric agent and a controlled release layer which comprises at least about 80% of the at least one antihistamine and at least about 80% of the at least one decongestant.

42. The bi-layered tablet of claim 41, wherein the at least one anticholinergic agent comprises methscopolamine, the at least one antihistamine comprises chlorpheniramine and the at least one decongestant comprises phenylephrine.

43. The bi-layered tablet of claim 42, wherein the tablet comprises from about 1 mg to about 3 mg of methscopolamine nitrate or an equivalent molar amount of another methscopolamine compound, from about 9 mg to about 15 mg of chlorpheniramine maleate or an equivalent molar amount of another chlorpheniramine compound and from about 30 mg to about 50 mg of phenylephrine hydrochloride or an equivalent molar amount of another phenylephrine compound.

44. The bi-layered tablet of claim 43, wherein the tablet comprises about 2 mg of methscopolamine nitrate, about 12 mg of chlorpheniramine maleate and about 40 mg of phenylephrine hydrochloride.

45. A method of reducing or eliminating over-drying of mucous membranes in a patient which is caused by a combined administration of an anticholinergic agent and an antihistamine, wherein the method comprises administering to the patient the dosage unit of claim 1.

46. The method of claim 45, wherein the at least one antihistamine provides a therapeutic effect for at least about 8 hours.

47. The method of claim 45, wherein the at least one anticholinergic agent provides a therapeutic effect for at least about 2 hours.

48. The method of claim 45, wherein the dosage unit further comprises at least one decongestant.

49. A method of alleviating a condition which can be alleviated by a combined administration of at least an antihistamine and an anticholinergic agent, wherein the method comprises administering the dosage unit of claim 1 to a subject in need thereof.

50. A process for making the solid pharmaceutical dosage unit of claim 1, wherein the method comprises preparing a first composition which comprises the at least one anticholinergic agent and a second composition which comprises the at least one antihistamine, and combining the first and the second compositions.

51. The process of claim 50, wherein the first and second compositions are combined by using a tablet press.
